SEQUENCE LISTING (49 sequences)

SEQ ID NO: 1 - DNA, 151 bp, Artificial Sequence
  ALA tRNA gene
taatacgactcactatagggagagccccctgatgagtccgtgaggacgaaacggtacccggtaccgtcggggctatagctcagctgggagagcgcctgctttgcacgcaggaggtctgcggttcgatcccgcatagctccaccaggaagct

SEQ ID NO: 2 - DNA, 151 bp, Artificial Sequence
  ARG tRNA gene
taatacgactcactatagggagaatgcctgatgagtccgtgaggacgaaacggtacccggtaccgtcgcatccgtagctcagctggatagagtactcggctacgaaccgagcggtcggaggttcgaatcctcccggatgcaccaggaagct

SEQ ID NO: 3 - DNA, 152 bp, Artificial Sequence
  ASN tRNA gene
taatacgactcactatagggagaagaggactgatgagtccgtgaggacgaaacggtacccggtaccgtctcctctgtagttcagtcggtagaacggcggactgttaatccgtatgtcactggttcgagtccagtcagaggagccaggaagct

SEQ ID NO: 4 - DNA, 152 bp, Artificial Sequence
  ASP tRNA gene
taatacgactcactatagggagagctccctgatgagtccgtgaggacgaaacggtacccggtaccgtcggagcggtagttcagtcggttagaatacctgcctgtcacgcagggggtcgcgggttcgagtcccgtccgttccgccaggaagct

SEQ ID NO: 5 - DNA, 148 bp, Artificial Sequence
  CYS tRNA gene
taatacgactcactatagggagacgccctgatgagtccgtgaggacgaaacggtacccggtaccgtcggcgcgttaacaaagcggttatgtagcggattgcaaatccgtctagtccggttcgactccggaacgcgcctccaggaagct

SEQ ID NO: 6 - DNA, 151 bp, Artificial Sequence
  GLN tRNA gene
taatacgactcactatagggagaaccccactgatgagtccgtgaggacgaaacggtacccggtaccgtctggggtatcgccaagcggtaaggcaccggattctgattccggcattccgaggttcgaatcctcgtaccccagccaggaagct

SEQ ID NO: 7 - DNA, 150 bp, Artificial Sequence
  GLU tRNA gene
taatacgactcactatagggagaggacctgatgagtccgtgaggacgaaacggtacccggtaccgtcgtccccttcgtctagaggcccaggacaccgccctttcacggcggtaacaggggttcgaatcccctaggggacgccaggaagct

SEQ ID NO: 8 - DNA, 151 bp, Artificial Sequence
  GLY tRNA gene
taatacgactcactatagggagacccgcctgatgagtccgtgaggacgaaacggtacccggtaccgtcgcgggaatagctcagttggtagagcacgaccttgccaaggtcggggtcgcgagttcgagtctcgtttcccgctccaggaagct

SEQ ID NO: 9 - DNA, 151 bp, Artificial Sequence
  HIS tRNA gene
taatacgactcactatagggagacaccctgatgagtccgtgaggacgaaacggtacccggtaccgtcggtggctatagctcagttggtagagccctggattgtgattccagttgtcgtgggttcgaatcccattagccaccccaggaagct

SEQ ID NO: 10 - DNA, 151 bp, Artificial Sequence
  MET tRNA gene
taatacgactcactatagggagacgcgctgatgagtccgtgaggacgaaacggtacccggtaccgtccgcggggtggagcagcctggtagctcgtcgggctcataacccgaaggtcgtcggttcaaatccggcccccgcaaccaggaagct

SEQ ID NO: 11 - DNA, 151 bp, Artificial Sequence
  MET tRNA gene
taatacgactcactatagggagaagccctgatgagtccgtgaggacgaaacggtacccggtaccgtcggctacgtagctcagttggttagagcacatcactcataatgatggggtcacaggttcgaatcccgtcgtagccaccaggaagct

SEQ ID NO: 12 - DNA, 152 bp, Artificial Sequence
  ILE tRNA gene
taatacgactcactatagggagaagcctctgatgagtccgtgaggacgaaacggtacccggtaccgtcaggcttgtagctcaggtggttagagcgcacccctgataagggtgaggtcggtggttcaagtccactcaggcctaccaggaagct

SEQ ID NO: 13 - DNA, 161 bp, Artificial Sequence
  LEU tRNA gene
taatacgactcactatagggagatcgcctgatgagtccgtgaggacgaaacggtacccggtaccgtcgcgaaggtggcggaattggtagacgcgctagcttcaggtgttagtgtccttacggacgtgggggttcaagtcccccccctcgcaccaggaagct

SEQ ID NO: 14 - DNA, 151 bp, Artificial Sequence
  LYS tRNA gene
taatacgactcactatagggagagacccctgatgagtccgtgaggacgaaacggtacccggtaccgtcgggtcgttagctcagttggtagagcagttgacttttaatcaattggtcgcaggttcgaatcctgcacgacccaccaggaagct

SEQ ID NO: 15 - DNA, 150 bp, Artificial Sequence
  PHE tRNA gene
taatacgactcactatagggagagggcctgatgagtccgtgaggacgaaacggtacccggtaccgtcgcccggatagctcagtcggtagagcaggggattgaaaatccccgtgtccttggttcgattccgagtccgggcaccaggaagct

SEQ ID NO: 16 - DNA, 151 bp, Artificial Sequence
  PRO tRNA gene
taatacgactcactatagggagaaccgctgatgagtccgtgaggacgaaacggtacccggtaccgtccggtgattggcgcagcctggtagcgcacttcgttcgggacgaaggggtcggaggttcgaatcctctatcaccgaccaggaagct

SEQ ID NO: 17 - DNA, 162 bp, Artificial Sequence
  SER tRNA gene
taatacgactcactatagggagacaccctgatgagtccgtgaggacgaaacggtacccggtaccgtcggtgaggtgtccgagtggctgaaggagcacgcctggaaagtgtgtatacggcaacgtatcgggggttcgaatccccccctcaccgccaggaagct

SEQ ID NO: 18 - DNA, 150 bp, Artificial Sequence
  THR tRNA gene
taatacgactcactatagggagacagcctgatgagtccgtgaggacgaaacggtacccggtaccgtcgctgatatggctcagttggtagagcgcacccttggtaagggtgaggtccccagttcgactctgggtatcagcaccaggaagct

SEQ ID NO: 19 - DNA, 152 bp, Artificial Sequence
  TRP tRNA gene
taatacgactcactatagggagagcccctctgatgagtccgtgaggacgaaacggtacccggtaccgtcaggggcgtagttcaattggtagagcaccggtctccaaaaccgggtgttgggagttcgagtctctccgcccctgccaggaagct

SEQ ID NO: 20 - DNA, 159 bp, Artificial Sequence
  TYR tRNA gene
taatacgactcactatagggagacaccctgatgagtccgtgaggacgaaacggtacccggtaccgtcggtggggttcccgagcggccaaagggagcagactgtaaatctgccgtcacagacttcgaaggttcgaatccttcccccaccaccaggaagct

SEQ ID NO: 21 - DNA, 151 bp, Artificial Sequence
  VAL tRNA gene
taatacgactcactatagggagacacccctgatgagtccgtgaggacgaaacggtacccggtaccgtcgggtgattagctcagctgggagagcacctcccttacaaggagggggtcggcggttcgatcccgtcatcacccaccaggaagct

SEQ ID NO: 22 - DNA, 20 bp, Artificial Sequence
  Forward tRNA
tgtcgccctttacacgtact

SEQ ID NO: 23 - DNA, 20 bp, Artificial Sequence
  Reverse Ala tRNA
tggtggagctatgcgggatc

SEQ ID NO: 24 - DNA, 18 bp, Artificial Sequence
  Reverse Arg tRNA
tggtgcatccgggaggat

SEQ ID NO: 25 - DNA, 18 bp, Artificial Sequence
  Reverse Asn tRNA
tggctcctctgactggac

SEQ ID NO: 26 - DNA, 18 bp, Artificial Sequence
  Reverse Asp tRNA
tggcggaacggacgggac

SEQ ID NO: 27 - DNA, 18 bp, Artificial Sequence
  Reverse Cys tRNA
tggaggcgcgttccggag

SEQ ID NO: 28 - DNA, 18 bp, Artificial Sequence
  Reverse Gln tRNA
tggctggggtacgaggat

SEQ ID NO: 29 - DNA, 18 bp, Artificial Sequence
  Reverse Glu tRNA
tggcgtcccctaggggat

SEQ ID NO: 30 - DNA, 18 bp, Artificial Sequence
  Reverse Gly tRNA
tggagcgggaaacgagac

SEQ ID NO: 31 - DNA, 18 bp, Artificial Sequence
  Reverse His tRNA
tggggtggctaatgggat

SEQ ID NO: 32 - DNA, 18 bp, Artificial Sequence
  Reverse fMet tRNA
tggttgcgggggccggat

SEQ ID NO: 33 - DNA, 17 bp, Artificial Sequence
  Reverse Met tRNA
ggtggctacgacgggat

SEQ ID NO: 34 - DNA, 18 bp, Artificial Sequence
  Reverse Ile tRNA
tggtaggcctgagtggac

SEQ ID NO: 35 - DNA, 18 bp, Artificial Sequence
  Reverse Leu tRNA
tggtgcgaggggggggac

SEQ ID NO: 36 - DNA, 18 bp, Artificial Sequence
  Reverse Lys tRNA
tggtgggtcgtgcaggat

SEQ ID NO: 37 - DNA, 18 bp, Artificial Sequence
  Reverse Phe tRNA
tggtgcccggactcggaa

SEQ ID NO: 38 - DNA, 21 bp, Artificial Sequence
  Reverse Pro tRNA
tggtcggtgatagaggattcg

SEQ ID NO: 39 - DNA, 18 bp, Artificial Sequence
  Reverse Ser tRNA
tggcggtgagggggggat

SEQ ID NO: 40 - DNA, 20 bp, Artificial Sequence
  Reverse Thr tRNA
tggtgctgatacccagagtc

SEQ ID NO: 41 - DNA, 18 bp, Artificial Sequence
  Reverse Trp tRNA
tggcaggggcggagagac

SEQ ID NO: 42 - DNA, 18 bp, Artificial Sequence
  Reverse Tyr tRNA
tggtgggggaaggattcg

SEQ ID NO: 43 - DNA, 18 bp, Artificial Sequence
  Reverse Val tRNA
tggtgggtgatgacggga

SEQ ID NO: 44 - DNA, 63 bp, Artificial Sequence
  Met-(Val)20
atggtagtagtagtagtagtagtagtagtagtagtagtagtagtagtagtagtagtagtagta

SEQ ID NO: 45 - protein, 21 aa, Artificial Sequence
  peptide sequence of Met-(Val)20
MVVVVVVVVVVVVVVVVVVVV

SEQ ID NO: 46 - protein, 11 aa, Artificial Sequence
  peptide sequence of Met-Tyr-(Val)9
MYVVVVVVVVV

SEQ ID NO: 47 - DNA, 33 bp, Artificial Sequence
  Met-Tyr-(Val)9
atgtacgtagtagtagtagtagtagtagtagta

SEQ ID NO: 48 - protein, 11 aa, Artificial Sequence
  peptide sequence of Met-pPa-(Val)9  (pPa is p-propargyloxyphenylalanine)
MXVVVVVVVVV

SEQ ID NO: 49 - DNA, 66 bp, Artificial Sequence
  ATG-NNN-(GTA) where “NNN” represents the codon being probed for degeneracy
atgnnngtagtagtagtagtagtagtagtagtagtagtagtagtagtagtagtagtagtagtagta

(19) United States
(12) Patent Application Publication
(10) Pub. No.: US 2016/0115487 A1
(43) Pub. Date: Apr. 28, 2016

(54) CELL-FREE SYNTHETIC INCORPORATION OF NON-NATURAL AMINO ACIDS INTO PROTEINS

(71) Applicants: Bradley C. BUNDY, Provo, UT (US); Mark T. SMITH, Pleasant Grove, UT (US); Jeffrey C. WU, Orem, UT (US); BRIGHAM YOUNG UNIVERSITY, Provo, UT (US)

(72) Inventors: Bradley C. Bundy, Provo, UT (US); Mark T. Smith, Pleasant Grove, UT (US); Jeffrey C. Wu, Orem, UT (US)

(21) Appl. No.: 14/894,638

(22) PCT Filed: May 29, 2014
(86) PCT No.: PCT/US14/40078

§ 371 Date: Nov. 30, 2015

Related U.S. Application Data
(60) Provisional application No. 61/855,983, filed on May 29, 2013.

(51) Int. Cl. C12N 15/67; C12P 21/02

(52) U.S. Cl. CPC C12N 15/67; C12P 21/02

(57) ABSTRACT

The present disclosure provides methods for incorporating at least one non-natural amino acid into a polypeptide using a cell-free protein synthesis which includes a cell-free extract and is deficient in endogenous tRNA. The methods include providing the synthesis system with at least one non-natural amino acid, at least one orthogonal tRNA and one orthogonal aminoacyl-tRNA synthetase which aminoacylates the corresponding orthogonal tRNA with the non-natural amino acid. The methods may also include removing the endogenous tRNA in the cell-free protein synthesis system by treating the synthesis system with a ribonuclease to degrade the endogenous tRNA and providing the synthesis system with a minimal set of tRNAs for one or more natural amino acids and the corresponding amino-acyl tRNA synthetases, such that the lack of some tRNA will enable unique codons to encode for non-natural amino acid incorporation with no or a minimal amount of completion from the tRNA present.

### CROSS REFERENCE TO RELATED APPLICATION

This application claims the benefit of the filing date under 35 U.S.C. §119(e) of U.S. Provisional Patent Application No. 61/855,983, filed on May 29, 2013, which is incorporated by reference herein in its entirety.

### FEDERALLY SPONSORED RESEARCH OR DEVELOPMENT

The present invention was made with support under award instrument number D13AP00037 awarded by the U.S. Department of Defense (Defense Advanced Research Projects Agency). The government has certain rights in this invention.

### FIELD OF THE TECHNOLOGY

The present disclosure generally relates to the field of protein engineering and protein biosynthesis, and more particularly to the fields of methods and compositions for producing proteins or polypeptides that incorporate non-natural amino acids.

### BACKGROUND

Engineering the incorporation of a non-natural amino acid (NNAA) with a chemically unique side chain at the desired location in the protein can selectively introduce unique chemical and physical properties to target proteins, while at the same time incorporation of a NNAA may also preserve the native structure and activity of the target proteins. In addition, non-natural amino acids add to the chemical diversity of protein, and thus can be used to create proteins with new functions. To date more than 50 non-natural amino acids have been incorporated into proteins produced in bacterial, yeast and mammalian systems.

Non-natural amino acid incorporation into proteins thus is a promising and rapidly advancing way to expand the language of biology, however, conventional technology has several limitations. These limitations include: 1) inefficient and inaccurate NNAA incorporation due to competition from native components; 2) inefficient and inaccurate NNAA incorporation due to the challenge of controlling and optimizing the concentrations and activities of the exogenous machinery necessary for NNAA incorporation; 3) limited protein yields at the laboratory scale; and 4) toxicity of the non-natural product and intermediates (including the NNAA itself) to a host organism.

Compared to in vivo expression, a cell-free protein synthesis system may provide many advantages such as, but not limited to, improved monitoring and control, reduced reaction volumes, virtually silenced background expression, simplified purification, and removal of the effect of many toxins. Accordingly, a need exists for a cell-free synthesis system to overcome the limitations of the in vivo expression and enable the incorporation of many different NNAAs. Particularly, there is a need to overcome the lack of naturally occurring unique codons that are not employed by endogenous machinery and that can be used to encode for NNAA incorporation.

### SUMMARY OF THE INVENTION

In one aspect, a method for incorporating at least one non-natural amino acid into a polypeptide is provided. The method uses a cell-free protein synthesis system which includes a cell-free extract and is deficient in endogenous tRNA. The method includes providing the cell-free protein synthesis system with the at least one non-natural amino acid, at least one orthogonal aminoacyl-tRNA synthetase and an orthogonal tRNA corresponding to the at least one non-natural amino acid. The orthogonal aminoacyl-tRNA synthetase aminoacylates the corresponding orthogonal tRNA with the non-natural amino acid. In some aspects, the methods include removing the endogenous tRNA in the cell-free protein synthesis system by treating the synthesis system with a ribonuclease to degrade the endogenous tRNA and providing the synthesis system with a set of tRNAs for one or more natural amino acids and the corresponding amino-acyl tRNA synthetases, wherein each of the one or more natural amino acids corresponds to a single unique codon.

In another aspect, a method for producing a set of tRNAs for one or more natural amino acids is provided. The method includes identifying gene sequences encoding for tRNAs for the one or more natural amino acids, and producing the tRNAs for the natural amino acids with RNA polymerase and tRNA gene templates that include the tRNA gene sequences.

In another aspect, a minimal set of tRNA gene sequences for one or more natural or non-natural amino acids is also provided. The tRNA gene sequences for one or more natural amino acids are set forth in SEQ ID NOs: 1-21 (Table 1).

In another aspect, a method for synthesizing a polypeptide is provided. The method includes providing at least one non-natural amino acid, at least one orthogonal aminoacyl-tRNA synthetase, and an orthogonal tRNA corresponding to the at least one non-natural amino acid, wherein the orthogonal aminoacyl-tRNA synthetase aminoacylates the corresponding orthogonal tRNA with the non-natural amino acid.

In yet another aspect, a method for synthesizing a polypeptide is provided. The method includes contacting a non-natural amino acid with an orthogonal aminoacyl-tRNA synthetase and an orthogonal tRNA. In some embodiments, the method is conducted in a cell-free environment. In some embodiments, the cell-free environment is deficient in endogenous tRNA.

Advantages of the present disclosure will become more apparent to those skilled in the art from the following description of embodiments and prophetic examples that have been shown and described by way of illustration. The invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 illustrates a method for incorporating at least one non-natural amino acid into a polypeptide using a cell-free system according to an embodiment of the present disclosure;

FIG. 2 shows a comparison of protein expression level as measured by fluorescence activity of produced green fluorescent protein (GFP) with different treatment time of RNAse immobilized on matrix material or beads and/or addition of 0.4 mg/mL and 2 mg/mL purified bulk tRNA: (1) untreated cell-free protein synthesis (CFPS) system as a control; (2) addition of protease inhibitor phenylmethylsulfonyl fluoride (PMSF) to untreated CFPS as a control; and (3)-(7) 5 min, 10 min, 20 min, 30 min and 60 min treatment of the CFPS system with RNAse immobilized on matrix material or beads;

FIG. 3 is a schematic representation of a tRNA gene template illustrating an embodiment of the design of the tRNA gene to synthesize tRNAs with an RNA polymerase in vitro or in a tRNA-free cell-free system;

FIG. 4 shows a gel electrophoresis pattern for PCR synthesized tranzyme tRNA via in vitro transcription: A) lanes from left to right: DNA ladder, negative and positive controls; B) lanes from left to right: DNA ladder, bulk purified tRNA, synthesized tranzyme tRNA from the T7-IVT (T7-In vitro transcription) reaction; Negative control.

FIG. 5 shows a comparison of cell-free protein synthesis level of C14-labeled polyvaline in a cell-free tRNA-depleted extract with 1) coexpression of orthogonal tranzyme DNA (otDNA); 2) no presence of tRNA; 3) addition of purified bulk E. coli tRNA;

FIG. 6 shows a comparison of protein expression level as measured by fluorescence activity of produced GFP with treatment of different recycled bead-immobilized RNAses for different incubation time;

FIG. 7 shows a comparison of protein expression level as measured by fluorescence activity of produced GFP with treatment of different multiple-recycled bead-immobilized RNAses and/or addition of purified bulk tRNA to the cell-free protein synthesis system with different incubation time; and

FIG. 8 shows a comparison of cell-free protein synthesis level of C14-labeled polyvaline in a cell-free tRNA-depleted extract with 1) left: change of concentration of Val otDNA as concentration of fMet otDNA was held constant; 2) right: change of concentration of fMet otDNA as concentration of Val otDNA was held constant. This demonstrates optimization of protein production yields in the cell-free protein synthesis system by optimizing the two different tranzyme tRNA gene template concentrations, both of which are needed for the production of Methionine-initiated polyvaline.

### DETAILED DESCRIPTION

The present disclosure relates to methods for incorporating non-natural amino acids into a polypeptide using a cell-free protein synthesis system, wherein the cell-free protein synthesis system is deficient in endogenous tRNA. The present disclosure also relates to methods for producing a set of tRNAs for one or more natural amino acids that can be used in the cell-free protein synthesis system. The present disclosure also describes tRNA gene sequences to synthesize both natural amino acids and non-natural amino acids in vivo and in a tRNA-free, cell-free system.

The practice of the methods disclosed herein will employ, unless otherwise indicated, conventional methods of virology, microbiology, molecular biology and recombinant DNA techniques within the skill of the art. Such techniques are explained fully in the literature. See, e.g. Calhoun and Swartz (2005) Biotechnol Bioeng 90(5):606-13; Jewett and Swartz (2004) Biotechnol Bioeng 86(1):19-26; Jewett et al. (2002) Prokaryotic Systems for In Vitro Expression. In: Weiner M, Lu Q, editors. Gene cloning and expression technologies. Westborough, Mass.: Eaton Publishing. p 391-411; Lin et al. (2005) Biotechnol Bioeng 89(2):148-56; Wang et al. (2001) Science 292(5516):498-500; Wang et al. (2003) Proc Natl Acad Sci USA 100(1):56-61; Chin et al. (2002) J Am Chem Soc 124(31):9026-7; Farrell et al. (2005) Nat Methods, 2005. 2(5):377-84; Liu et al. (2003) J Am Chem Soc 125(7):1702-3; Albayrak and Swartz (2013) Nucleic Acids Res 41(11):5949-5963; Fechter et al. (1998) FEBS Letters 436(1):99-103; Smith et al. (2014) Protein Biochem. 49(2): 217-222; Shrestha et al. (2014) New Biotech. 31(1):28-34. All references cited herein are incorporated by reference.

In some embodiments, the practice of methods disclosed herein will employ, unless otherwise indicated, conventional methods of virology, microbiology, molecular biology, recombinant DNA techniques, and RNA transcription by in vitro transcription within the skill of the art. Such techniques are explained fully in the literature. See, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.) All references cited herein are incorporated by reference.

### DEFINITIONS

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

The terms “protein,” “peptide,” and “polypeptide” are used interchangeably herein.

As used herein, the term “orthogonal” refers to a molecule (e.g., an orthogonal tRNA (O-tRNA) and or an orthogonal aminoacyl tRNA synthetase (O-RS)) that is used with reduced efficiency by a system of interest (e.g., a translational system, e.g., a cell). Orthogonal refers to the inability or reduced efficiency, e.g., less than 20% efficient, less than 10% efficient, less than 5% efficient, or e.g., less than 1% efficient, of an O-tRNA and/or O-RS to function in the translation system of interest. An orthogonal molecule may function independently of or without interactions with the synthetases and tRNAs endogenous to the system of interest. For example, an O-tRNA in a translation system of interest aminoacylates any endogenous RS of a translation system of interest with reduced or even zero efficiency, when compared to aminoacylation of an endogenous tRNA by the endogenous RS. In another example, an O-RS aminoacylates any endogenous tRNA in the translation system of interest with reduced or even zero efficiency, as compared to aminoacylation of the endogenous tRNA by an endogenous RS.

The term “preferentially aminoacylates” refers to an efficiency of, e.g., about 70% efficient, about 75% efficient, about 85% efficient, about 90% efficient, about 95% efficient, or about 99% or more efficient, at which an O-RS aminoacylates an O-tRNA with an unnatural amino acid compared to a naturally occurring tRNA or starting material used to generate the O-tRNA. The unnatural amino acid is then incorporated into a growing polypeptide chain with high fidelity, e.g., at greater than about 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

“Non-specific base pairing” refers to any base pairing between the tRNA anticodon loop and amino acids that is weaker than the Watson-Crick base-pairing. In most cases, except Met and Trp, a natural amino acid is encoded by two or more genetic codes (i.e. degenerate genetic codes). To recognize all the degenerate genetic codes for the natural amino acid, the anticodon loop of the wild-type tRNA(s) relies on both promiscuous base-pairing (e.g. wobble base-paring) and pure Watson-Crick base-pairing. Non-specific base pairing refers to base pairing between the tRNA anticodon loop and amino acids that is weaker than the Watson-Crick base-pairing. Non-specific base pairing may be facilitated by post-transcriptional modifications known in the art.

“Standard,” “canonical,” or “natural” amino acids are the 20 proteinogenic alpha-amino acids that in nature are the building-blocks of proteins within all domains of life, and that are also directly encoded by the genetic code. All others are known as “non-standard,” “non-canonical,” “non-natural” or “unnatural.”

As used herein, the terms “non-natural amino acid,” “NNAA,” and “unnatural amino acid” interchangeably refer to any amino acid, modified amino acid, and/or amino acid analogue that is not one of the 20 naturally occurring amino acids or selenocysteine. Examples of unnatural amino acids that can be used include but not limited to: an unnatural analogue tyrosine, glutamine, phenylalanine, serine, threonine; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; a cyclic amino acid other than proline, etc. For example, the unnatural amino acid can be an O-methyl-L-tyrosine, an L-3-(2-naphthyl)alanine, a 3-methyl-phenylalanine, an 0-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine.in one embodiment, the at least one unnatural amino acid is an O-methyl-L-tyrosine. In one specific example embodiment, the at least one unnatural amino acid is an L-3-(2-naphthyl)alanine. In another set of specific examples, the at least one unnatural amino acid is an amino-, isopropyl-, or O-allyl-containing phenylalanine analogue.

As used herein, the term “orthogonal components” refers to an exogenous orthogonal aminoacyl-tRNA synthetase (O-RS), an exogenous O-tRNA (i.e. aminoacylated), and a NNAA which works in concert to charge the O-tRNA with the NNAA. The exogenous O-RS/O-tRNA pair is mutated to exclusively recognize and charge the corresponding NNAA to the exogenous tRNA. Such components are known in the art, for example as described in U.S. Pat. No. 7,045,337, issued May 16, 2006, incorporated herein by this reference. The orthogonal tRNA recognizes a selector codon, which may be nonsense codons such as, stop codons, e.g., amber, ochre, and opal codons; or four or more base codons; codons derived from natural or unnatural base pairs and the like. In almost all cases there is competition from endogenous components for the selector codon. The orthogonal tRNA anticodon loop recognizes the selector codon on the mRNA and incorporates the NNAA at this site in the polypeptide.

As used herein, the term “tranzyme” refers to a ribozyme-tRNA complex.

The term “cell-free protein synthesis” as used herein refers to an in vitro protein synthesis using a cell extract. This may be protein synthesis in a cell-free translation system for producing proteins on ribosome through reading of information of mRNA or protein synthesis in a coupled system comprising a cell-free transcription system that produces mRNA using DNA as a template and a cell-free translation system that translates the mRNA information into proteins. When DNA is used for a template, various kinds of template DNA can be prepared simultaneously and rapidly by the amplification reaction in vitro such as Polymerase Chain Reaction (PCR). The cell extracts may be derived from any eukaryotic or prokaryotic cells, such as E. coli, yeast, wheat germ extract, rabbit reticulocyte cells, insect cells, animal cells and human cell lines etc.

The term “derived from” refers to a component that is isolated from an organism or isolated and modified, or generated, e.g., chemically synthesized, using information of the component from the organism.

The term “deficient in endogenous tRNA” as used herein refers to a cell-free protein synthesis system where the endogenous tRNA has been removed or degraded to a minimal amount that the endogenous tRNA cannot compete with the exogenous tRNA.

Unless otherwise defined herein or below in the remainder of the specification, all technical and scientific terms used herein have the same meaning as commonly understood by those of ordinary skill in the art to which the invention belongs.

Cell-Free Protein Synthesis Systems

The cell-free protein synthesis systems (CFPS), also known as in vitro protein production systems, for the present disclosure may be cell-free translation systems based on cell extracts. In some embodiments, a cell-free protein synthesis system which is deficient in endogenous tRNA is used. The CFPS may also include a combination of biomachinery from different organisms to create synthetic pathways and products known by a person skilled in the art.

In some embodiments, the CFPS is a combined reaction mix of biomachinery from different biological extracts and/or defined reagents. The reaction mix will comprise a template for production of the macromolecule, e.g. DNA, mRNA, etc.; monomers for the macromolecule to be synthesized, e.g. non-natural amino acids, nucleotides, etc., and such co-factors, enzymes and other reagents that are necessary for the synthesis, e.g. ribosomes, polymerases, transcriptional factors, etc., except the endogenous tRNA, which can be removed from the biological extracts. In some embodiments, the tRNA for either non-natural amino acids or natural amino acids or a combination of both, can be synthesized exogenously, purified and added to the reaction mix of the invention. Alternatively, the tRNAs can be synthesized in the reaction mix. The cell free synthesis reaction may be performed as batch, continuous flow, semi-continuous flow, continuous exchange, or other methods as known in the art.

Cell extracts as used herein may be any preparation comprising the components required for protein synthesis machinery. The cell-free protein synthesis system may include extracts from eukaryotic or prokaryotic cells, such as E-coli, yeast, wheat germ extract, rabbit reticulocyte cells, insect cells, animal cells and human cell lines etc. By way of non-limiting example, a bacterial cell extract that includes components capable of expressing a nucleic acid encoding a desired protein may be used. Thus, a bacterial extract includes components that are capable of translating messenger ribonucleic acid (mRNA) encoding a desired protein, and optionally includes components that are capable of transcribing DNA encoding a desired protein. Such components include, for example, DNA-directed RNA polymerase (RNA polymerase), any transcription activators that are required for initiation of transcription of DNA encoding the desired protein, aminoacyl-tRNA synthetases, 70S ribosomes, N10-formyltetrahydrofolate, formylmethionine-tRNAfMet synthetase, peptidyl transferase, initiation factors such as IF-1, IF-2 and IF-3, elongation factors such as EF-Tu, EF-Ts, and EF-G, release factors such as RF-1, RF-2, and RF-3, and the like. In some embodiments, the transfer ribonucleic acids (tRNAs) in the cell extracts can be removed.

In some embodiments, the reaction mixture includes extracts from bacterial cells, e.g. E. coli S12 extracts. In some embodiments, the bacterial cells are modified such that the bacterial cells endogenously express an orthogonal tRNA. The organism used as a source of extracts may be referred to as the source organism. Exemplary methods for producing active extracts may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York; Kudlicki et al. (1992) Anal Biochem 206(2):389-93, incorporated herein by reference.

Examples of cell extracts as used herein were based on crude extracts prepared from Escherichia coli (E. coli) (Shrestha et al. (2014) New Biotech. 31(1):28-34). In some embodiments, the bacterial strain from which the extract is derived may be further modified. By way of non-limiting example, the E. coli-based CFPS may be modified to decrease background gene expression and increase protein production to levels to exceed 1 mg/mL (Liu et al. (2005) Biotechnol Progr. 21:460-5; Yang et al. (2012) Biotechnol Progr. 28:413-20), incorporated by reference.

Orthogonal Aminoacyl-tRNA Synthetases

Orthogonal aminoacyl-tRNA Synthetases (O-RS) are enzymes that charge (acylate) tRNAs with non-natural samino acids. These charged aminoacyl tRNAs then participate in mRNA translation and protein synthesis. The O-RS show high specificity for charging a specific orthogonal tRNA with the appropriate non-natural amino acid. For example, valyl-tRNA with valine by valyl-tRNA synthetase, or tryptophanyl-tRNA with tryptophan by tryptophanyl-tRNA synthetase. The non-natural O-RS and tRNA pairs used in the present disclosures as examples are O-RS derived from Methanocaldococcus jannaschii and Tyrosyl derivative as described in U.S. Pat. No. 7,713,721, issued May 11, 2010; Methanosarcina barkeri and Pyrrolysyl (Genbank Accession Number: AY273828.1); and Saccharomyces cerevisiae and Tryptophanyl (modified based on NCBI Accession Number NM_001183351.1, Chatterjee et al. (2013) Angewandte Chemie Int. Ed. 52(19): 5106-09), all incorporated by reference.

Orthogonal tRNA synthetase can be synthesized exogenously, purified and added to the reaction mix. In some embodiments, the orthogonal tRNA synthetase as added to the reaction mix in a defined quantity, of at least about 10 μg/ml, at least about 20 μg/ml, at least about 30 μg/ml. The protein may be synthesized in bacterial or eukaryotic cells and purified, e.g. by affinity chromatography, PAGE, gel exclusion chromatography, reverse phase chromatography, and the like, as known in the art.

In some embodiments, the E. coli-orthogonal synthetase can be produced in vivo and purified, as previously described Smith et al. (2013) Biotechnol Prog. 29:247-54 incorporated by reference.

The cell-free protein synthesis system described herein provides the ability to synthesize proteins that comprise unnatural amino acids in usefully large quantities. For example, proteins comprising at least one unnatural amino acid can be produced at a concentration of at least about 10, 50, 100 or more micrograms per liter, e.g., in a composition comprising a cell extract, a buffer, a pharmaceutically acceptable excipient, and/or the like.

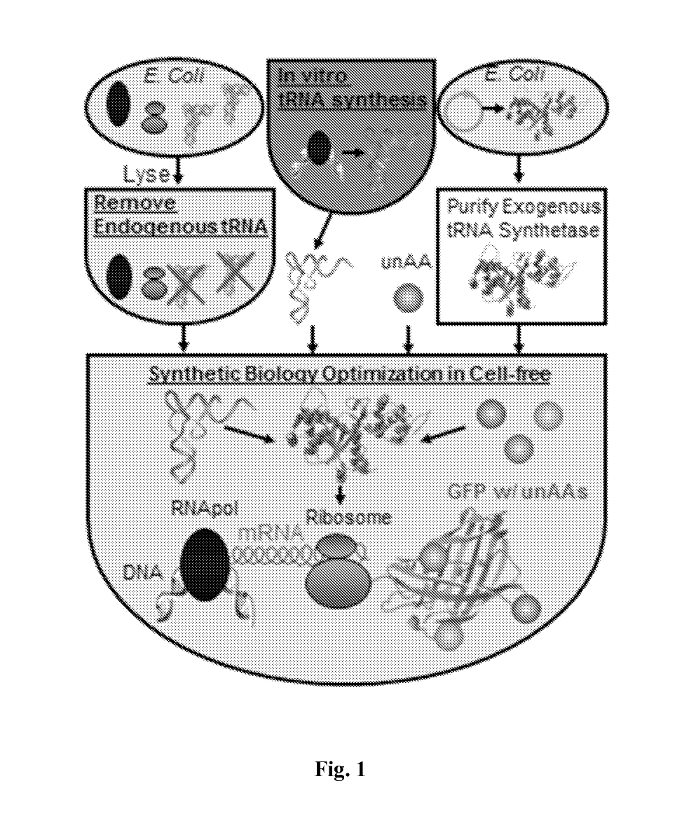

As shown in FIG. 1, the present disclosure provides a method for incorporating at least one NNAA into a protein using a cell-free system. The protein synthesized with the methods disclosed herein can be either a polypeptide with both natural amino acids and non-natural amino acids incorporated. In some embodiments, the protein synthesized according to the method described herein can be an entirely synthetic protein construct which incorporates non-natural amino acids only. The cell-free synthesis reaction may involve both orthogonal tRNA/orthogonal aminoacyl-tRNA synthetase and natural tRNAs/natural aminoacyl-tRNA synthetase for natural amino acids.

In some aspects, the methods include removing the endogenous tRNA in the cell-free protein synthesis system by treating the synthesis system with a ribonuclease to degrade the endogenous tRNA and providing the synthesis system with a set of tRNAs for one or more natural amino acids and the corresponding amino-acyl tRNA synthetases, wherein each of the one or more natural amino acids corresponds to a single unique codon. In some embodiments, the ribonuclease can be RNase A, Onconase, Colicin D tRNase or other ribonuclease depending on the types of cell extract used for the cell-free synthesis reaction.

In some embodiments, the tRNAs for either the NNAAs or natural amino acids or both can be synthesized in the cell-free protein synthesis system. In some embodiments, these tRNAs can also be synthesized exogenously and added to the cell-free protein synthesis system.

In some embodiments, both the orthogonal tRNAs and tRNAs for natural amino acids can be made with an RNA polymerase and a tRNA gene template comprising the gene sequences of the tRNA. In some embodiments, the tRNA gene template can be a linear expression template. The design of the synthetic tRNA can be produced such that the 5′ and 3′ ends of the final RNA product will include the appropriate termini for tRNA. In some aspects, the linear tRNA templates can be generated using single-step or two-step PCR. In some embodiments, a hammerhead ribozyme cleaves the 5′ end appropriately, while the 3′ end is digested prior to transcription to allow the T7 RNAP to terminate transcription by releasing the DNA rather than using a T7 terminator sequence. In some embodiments, the linear tRNA template can also terminate RNA-polymerase action by ending the tRNA-gene abruptly at the 3′ terminus of the tRNA gene.

In some embodiments, a concentration ratio of the tRNAs and tRNA gene template can also be optimized to increase efficiency of the non-natural amino acid incorporation.

In some embodiments, the codons for the non-natural amino acids can be selected based on the codons of the one or more natural amino acids so that the competition between the natural amino acids and the non-natural amino acids to be attached to the orthogonal tRNAs is reduced. In some aspect, the codons for the non-natural amino acids can be selected based on frequency of codon use, frequency of being incorporated by natural tRNAs, and predicted promiscuity patterns.

In some embodiments, a minimal set of tRNA for natural amino acids can also be used to emancipate codons for unnatural amino acid incorporation. In some embodiments, the tRNA for one or more natural or non-natural amino acids can be obtained through the steps: 1) identifying gene sequences encoding for tRNAs for the one or more natural or non-natural amino acids; and 2) producing the tRNAs for the natural or non-natural amino acids with an RNA polymerase and a tRNA gene template comprising the gene sequences. In some embodiments, the gene sequences encoding for tRNAs that form wobble base pairing or other non-specific base pairing with the codons of natural amino acids can be removed.

In some embodiments, methods for incorporating one or more non-natural amino acid into a polypeptide can further include selecting a concentration of the orthogonal tRNA and the orthogonal aminoacyl-tRNA synthetase to increase efficiency of the non-natural amino acid incorporation. In some aspects, the concentration of the non-natural amino acid can also be optimized to increase efficiency of the non-natural amino acid incorporation.

In some aspects, the present invention provides a kit for producing a polypeptide with at least one non-natural amino acid incorporated into the polypeptide, comprising a cell-free protein synthesis system wherein endogenous tRNA is removed, at least one non-natural amino acid, at least one orthogonal aminoacyl-tRNA synthetase and its corresponding orthogonal tRNA, wherein the orthogonal aminoacyl-tRNA synthetase preferentially aminoacylates the corresponding orthogonal tRNA with the non-natural amino acid.

### EXAMPLES

The following examples serve to illustrate the invention without limiting the invention in its scope.

### Example 1

### Methods for Removing Endogenous tRNA in the Cell-Free Protein Synthesis System

Extract Pretreatment: Escherichia coli strain BL21 Star DE3™ (Invitrogen, USA) cells were used to prepare the extract. The cell extract can be prepared, aliquoted, and flash-frozen in liquid nitrogen for storage at −80 degrees Celsius. (Calhoun et al. (2005) Biotechnol Progr. 21:1146-53). Prior to use, the cell extract was thawed on ice and subsequently centrifuged at 16000×g for 10 minutes. The resulting supernatant was used in the CFPS system described herein.

In an exemplary embodiment, RNAse A columns with RNAses immobilized on matrix material or Superparamagnetic beads were used to degrade the tRNAs in an E. coli cell-free extract. In some embodiments, the ribonuclease which is immobilized on the beads can also be recycled.

RNAse A Immobilization on Superparamagnetic Beads: Bovine pancreas RNAse A (Sigma Aldrich, USA) was incubated with epoxy-functionalized M-270 Dynabeads (Life Technologies, USA) in the presence of ammonium sulfate. Reaction concentrations were performed in a 0.1 M sodium phosphate buffer pH 7.4 as follows: 6.06 μg/mL RNAse A, 2 billion beads per mL, and 3 M ammonium sulfate. Reactions volumes of 0.2 mL were tilted end-over-end at room temperature for 24 hours. Following the reaction, the RNAse-immobilized superparamagnetic beads were aggregated using a magnetic field and the reaction supernatant was removed. The RNAse-immobilized superparamagnetic beads were suspended, washed, and re-aggregated 3 times with 0.2 mL PBS pH 7.4 containing 0.5% v/v Tween2® and 1 time in 0.2 mL PBS. Finally, the beads were resuspended in 0.2 mL PBS and stored at 4 degrees Celsius until use.

Extract RNAse Treatment: To inhibit protease activity, 500 μM protease inhibitor PMSF was placed into the thawed, pretreated extracts and incubated for at least 5 minutes. In an exemplary embodiment, treatment reactions of the extracts with RNase were performed as follows: 70 million RNAse-decorated beads were added to 500 μL extract and incubated for at least 5, 10, 20, 30 or 60 minutes. Following incubation, beads were aggregated using a magnetic field and the treated extract was removed.

Cell-free Protein Synthesis (CFPS): A super-folder green fluorescent protein (GFP) variant was expressed in CFPS using treated extracts and untreated extracts as controls. CFPS was performed using a PANOx system or a modified PANOx-SP system (Jewett et al. (2004) Biotechnol Bioeng. 86:19-26) as follows: 20 μL reactions were incubated in sealed 96-well plates for 3 hours at 37 degrees Celsius. Reactions contained 25% v/v S12 Extract, 12 nmol/L pY71-sfGFP, 1.2 mM ATP, 86 mM each of GTP, CTP, and UTP, 1 mM 1,4-diaminobutane, 1.5 mM spermadine, 0.33 NAD, 0.27 mM CoA, 0.17 mM folinic acid, 2.7 mM potassium oxalate, 175 mM potassium glutamate, 10 mM ammonium glutamate, 33.33 mM phosphoenolpyruvate, and 9 mM magnesium glutamate. Bulk purified E. coli tRNA (Roche Applied Sciences, USA) was added to CFPS reactions as indicated. Quantifying extract Viability: GFP was expressed as a reported protein. The fluorescence level at the end of the CFPS reaction was used as a relative comparison of overall extract viability to produce GFP.

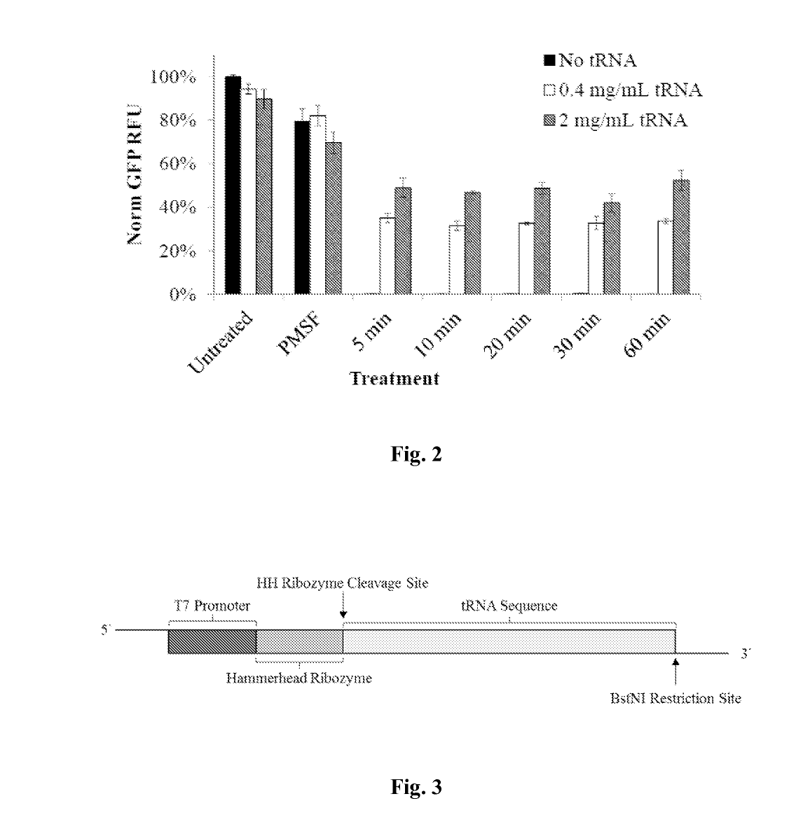

As shown in FIG. 2, treating a CFPS system with RNAse immobilized on matrix material or beads removes the endogenous tRNA. Also, addition of purified bulk E. coli tRNA to the CFPS system with untreated extract maintains protein production capability albeit with a slight decrease in protein yield. Addition of PMSF to otherwise untreated extracts maintains protein production capability albeit with a slightly decreased overall protein yield. Extracts treated with RNAse-decorated beads yielded undetectable levels of protein production as measured by fluorescence. Also shown in FIG. 2, addition of purified bulk tRNA to the treated extracts restored in part the extract's viability, which was measured by produced protein green fluorescent protein (GFP).

### Example 2

### In Vitro Synthesis of tRNA

The protein synthesized with the methods disclosed herein can be either a polypeptide with both natural amino acids and non-natural amino acids incorporated or can be an entirely synthetic protein construct which incorporate non-natural amino acids only.

In some embodiments, both the orthogonal tRNAs and tRNAs for natural amino acids can be made with an RNA polymerase and a tRNA gene template comprising the gene sequences of the tRNA. In some embodiments, as shown in FIG. 3, the tRNA gene template can be a linear expression template. The synthetic tRNA genes can be designed to produce the final RNA product with appropriate 5′ and 3′ termini for tRNA. The linear tRNA templates can be generated de novo with the desired anti-codon using a single-step or two-step PCR. In some embodiments, a hammerhead ribozyme cleaves the 5′ end appropriately, while the 3′ end is digested prior to transcription to allow the T7 RNAP to terminate transcription by releasing the DNA rather than using a T7 terminator sequence. In some embodiments, the linear tRNA template can also terminate RNA-polymerase action by ending the tRNA-gene abruptly at the 3′ terminus of the tRNA gene.

The tRNA gene template used as an example for the present invention was generated by custom synthesis and the linear tRNA DNA template was further amplified by PCR in preparation for cell-free protein synthesis. Plasmid and linear tRNA template were used immediately or stored in ddH₂O at −20° C. until use. The primer sequences used for generating linear tRNA template are tabulated in Table 2 below (SEQ ID NOs 22-43).

Exemplary sequences for the tDNAs in the linear tRNA templates are listed in Table 1. The tDNAs may include a hammerhead ribozyme and the corresponding tRNA sequence. The sequences of the linear tRNA templates for the 20 natural amino acids and the initiation codon (formyl-methionine, fMet) are listed in Table 1 (SEQ ID NOs 1-21).

TABEL 1

| Amino | | |
|---|---|---|
| Acid | tDNA | SEQ ID # |
| Ala | TAATACGACTCACTATAGGGAGAGCCCCCTGATGAGTCC | SEQ ID NO: 1 |
| | GTGAGGACGAAACGGTACCCGGTACCGTCGGGGCTATA | |
| | GCTCAGCTGGGAGAGCGCCTGCTTTGCACGCAGGAGGTC | |
| | TGCGGTTCGATCCCGCATAGCTCCACCAGGAAGCT | |
| Arg | TAATACGACTCACTATAGGGAGAATGCCTGATGAGTCCG | SEQ ID NO: 2 |
| | TGAGGACGAAACGGTACCCGGTACCGTCGCATCCGTAG | |
| | CTCAGCTGGATAGAGTACTCGGCTACGAACCGAGCGGTC | |
| | GGAGGTTCGAATCCTCCCGGATGCACCAGGAAGCT | |
| Asn | TAATACGACTCACTATAGGGAGAAGAGGACTGATGAGT | SEQ ID NO: 3 |
| | CCGTGAGGACGAAACGGTACCCGGTACCGTCTCCTCTGT | |
| | AGTTCAGTCGGTAGAACGGCGGACTGTTAATCCGTATGT | |
| | CACTGGTTCGAGTCCAGTCAGAGGAGCCAGGAAGCT | |
| Asp | TAATACGACTCACTATAGGGAGAGCTCCCTGATGAGTCC | SEQ ID NO: 4 |
| | GTGAGGACGAAACGGTACCCGGTACCGTCGGAGCGGTA | |
| | GTTCAGTCGGTTAGAATACCTGCCTGTCACGCAGGGGGT | |
| | CGCGGGTTCGAGTCCCGTCCGTTCCGCCAGGAAGCT | |
| Cys | TAATACGACTCACTATAGGGAGACGCCCTGATGAGTCCG | SEQ ID NO: 5 |
| | TGAGGACGAAACGGTACCCGGTACCGTCGGCGCGTTAA | |
| | CAAAGCGGTTATGTAGCGGATTGCAAATCCGTCTAGTCC | |
| | GGTTCGACTCCGGAACGCGCCTCCAGGAAGCT | |
| Gln | TAATACGACTCACTATAGGGAGAACCCCACTGATGAGTC | SEQ ID NO: 6 |
| | CGTGAGGACGAAACGGTACCCGGTACCGTCTGGGGTAT | |
| | CGCCAAGCGGTAAGGCACCGGATTCTGATTCCGGCATTC | |
| | CGAGGTTCGAATCCTCGTACCCCAGCCAGGAAGCT | |
| Glu | TAATACGACTCACTATAGGGAGAGGACCTGATGAGTCC | SEQ ID NO: 7 |
| | GTGAGGACGAAACGGTACCCGGTACCGTCGTCCCCTTCG | |
| | TCTAGAGGCCCAGGACACCGCCCTTTCACGGCGGTAACA | |
| | GGGGTTCGAATCCCCTAGGGGACGCCAGGAAGCT | |
| Gly | TAATACGACTCACTATAGGGAGACCCGCCTGATGAGTCC | SEQ ID NO: 8 |
| | GTGAGGACGAAACGGTACCCGGTACCGTCGCGGGAATA | |
| | GCTCAGTTGGTAGAGCACGACCTTGCCAAGGTCGGGGTC | |
| | GCGAGTTCGAGTCTCGTTTCCCGCTCCAGGAAGCT | |
| His | TAATACGACTCACTATAGGGAGACACCCTGATGAGTCCG | SEQ ID NO: 9 |
| | TGAGGACGAAACGGTACCCGGTACCGTCGGTGGCTATA | |
| | GCTCAGTTGGTAGAGCCCTGGATTGTGATTCCAGTTGTC | |
| | GTGGGTTCGAATCCCATTAGCCACCCCAGGAAGCT | |
| fMet | TAATACGACTCACTATAGGGAGACGCGCTGATGAGTCCG | SEQ ID NO: 10 |
| | TGAGGACGAAACGGTACCCGGTACCGTCCGCGGGGTGG | |
| | AGCAGCCTGGTAGCTCGTCGGGCTCATAACCCGAAGGTC | |
| | GTCGGTTCAAATCCGGCCCCCGCAACCAGGAAGCT | |
| Met | TAATACGACTCACTATAGGGAGAAGCCCTGATGAGTCCG | SEQ ID NO: 11 |
| | TGAGGACGAAACGGTACCCGGTACCGTCGGCTACGTAG | |
| | CTCAGTTGGTTAGAGCACATCACTCATAATGATGGGGTC | |
| | ACAGGTTCGAATCCCGTCGTAGCCACCAGGAAGCT | |
| Ile | TAATACGACTCACTATAGGGAGAAGCCTCTGATGAGTCC | SEQ ID NO: 12 |
| | GTGAGGACGAAACGGTACCCGGTACCGTCAGGCTTGTA | |
| | GCTCAGGTGGTTAGAGCGCACCCCTGATAAGGGTGAGG | |
| | TCGGTGGTTCAAGTCCACTCAGGCCTACCAGGAAGCT | |
| Leu | TAATACGACTCACTATAGGGAGATCGCCTGATGAGTCCG | SEQ ID NO: 13 |
| | TGAGGACGAAACGGTACCCGGTACCGTCGCGAAGGTGG | |
| | CGGAATTGGTAGACGCGCTAGCTTCAGGTGTTAGTGTCC | |
| | TTACGGACGTGGGGGTTCAAGTCCCCCCCCTCGCACCAG | |
| | GAAGCT | |
| Lys | TAATACGACTCACTATAGGGAGAGACCCCTGATGAGTCC | SEQ ID NO: 14 |
| | GTGAGGACGAAACGGTACCCGGTACCGTCGGGTCGTTA | |
| | GCTCAGTTGGTAGAGCAGTTGACTTTTAATCAATTGGTC | |
| | GCAGGTTCGAATCCTGCACGACCCACCAGGAAGCT | |
| Phe | TAATACGACTCACTATAGGGAGAGGGCCTGATGAGTCC | SEQ ID NO: 15 |
| | GTGAGGACGAAACGGTACCCGGTACCGTCGCCCGGATA | |
| | GCTCAGTCGGTAGAGCAGGGGATTGAAAATCCCCGTGTC | |
| | CTTGGTTCGATTCCGAGTCCGGGCACCAGGAAGCT | |
| Pro | TAATACGACTCACTATAGGGAGAACCGCTGATGAGTCCG | SEQ ID NO: 16 |
| | TGAGGACGAAACGGTACCCGGTACCGTCCGGTGATTGG | |
| | CGCAGCCTGGTAGCGCACTTCGTTCGGGACGAAGGGGTC | |
| | GGAGGTTCGAATCCTCTATCACCGACCAGGAAGCT | |
| Ser | TAATACGACTCACTATAGGGAGACACCCTGATGAGTCCG | SEQ ID NO: 17 |
| | TGAGGACGAAACGGTACCCGGTACCGTCGGTGAGGTGT | |
| | CCGAGTGGCTGAAGGAGCACGCCTGGAAAGTGTGTATA | |
| | CGGCAACGTATCGGGGGTTCGAATCCCCCCCTCACCGCC | |
| | AGGAAGCT | |
| Thr | TAATACGACTCACTATAGGGAGACAGCCTGATGAGTCCG | SEQ ID NO: 18 |
| | TGAGGACGAAACGGTACCCGGTACCGTCGCTGATATGG | |
| | CTCAGTTGGTAGAGCGCACCCTTGGTAAGGGTGAGGTCC | |
| | CCAGTTCGACTCTGGGTATCAGCACCAGGAAGCT | |
| Trp | TAATACGACTCACTATAGGGAGAGCCCCTCTGATGAGTC | SEQ ID NO: 19 |
| | CGTGAGGACGAAACGGTACCCGGTACCGTCAGGGGCGT | |
| | AGTTCAATTGGTAGAGCACCGGTCTCCAAAACCGGGTGT | |
| | TGGGAGTTCGAGTCTCTCCGCCCCTGCCAGGAAGCT | |
| Tyr | TAATACGACTCACTATAGGGAGACACCCTGATGAGTCCG | SEQ ID NO: 20 |
| | TGAGGACGAAACGGTACCCGGTACCGTCGGTGGGGTTC | |
| | CCGAGCGGCCAAAGGGAGCAGACTGTAAATCTGCCGTC | |
| | ACAGACTTCGAAGGTTCGAATCCTTCCCCCACCACCAGG | |
| | AAGCT | |
| Val | TAATACGACTCACTATAGGGAGACACCCCTGATGAGTCC | SEQ ID NO: 21 |
| | GTGAGGACGAAACGGTACCCGGTACCGTCGGGTGATTA | |
| | GCTCAGCTGGGAGAGCACCTCCCTTACAAGGAGGGGGT | |
| | CGGCGGTTCGATCCCGTCATCACCCACCAGGAAGCT | |

TABLE 2

| Description | PCR Primer Sequence | SEQ ID # |
|---|---|---|
| Forward tRNA | TGT CGC CCT TTA CAC GTA CT | SEQ ID NO: 22 |
| Reverse Ala tRNA | TGG TGG AGC TAT GCG GGA TC | SEQ ID NO: 23 |
| Reverse Arg tRNA | TGG TGC ATC CGG GAG GAT | SEQ ID NO: 24 |
| Reverse Asn tRNA | TGG CTC CTC TGA CTG GAC | SEQ ID NO: 25 |
| Reverse Asp tRNA | TGG CGG AAC GGA CGG GAC | SEQ ID NO: 26 |
| Reverse Cys tRNA | TGG AGG CGC GTT CCG GAG | SEQ ID NO: 27 |
| Reverse Gln tRNA | TGG CTG GGG TAC GAG GAT | SEQ ID NO: 28 |
| Reverse Glu tRNA | TGG CGT CCC CTA GGG GAT | SEQ ID NO: 29 |
| Reverse Gly tRNA | TGG AGC GGG AAA CGA GAC | SEQ ID NO: 30 |
| Reverse His tRNA | TGG GGT GGC TAA TGG GAT | SEQ ID NO: 31 |
| Reverse fMet tRNA | TGG TTG CGG GGG CCG GAT | SEQ ID NO: 32 |
| Reverse Met tRNA | GG TGG CTA CGA CGG GAT | SEQ ID NO: 33 |
| Reverse Ile tRNA | TGG TAG GCC TGA GTG GAC | SEQ ID NO: 34 |
| Reverse Leu tRNA | TGG TGC GAG GGG GGG GAC | SEQ ID NO: 35 |
| Reverse Lys tRNA | TGG TGG GTC GTG CAG GAT | SEQ ID NO: 36 |
| Reverse Phe tRNA | TGG TGC CCG GAC TCG GAA | SEQ ID NO: 37 |
| Reverse Pro tRNA | TGG TCG GTG ATA GAG GAT TCG | SEQ ID NO: 38 |
| Reverse Ser tRNA | TGG CGG TGA GGG GGG GAT | SEQ ID NO: 39 |
| Reverse Thr tRNA | TGG TGC TGA TAC CCA GAG TC | SEQ ID NO: 40 |
| Reverse Trp tRNA | TGG CAG GGG CGG AGA GAC | SEQ ID NO: 41 |
| Reverse Tyr tRNA | TGG TGG GGG AAG GAT TCG | SEQ ID NO: 42 |
| Reverse Val tRNA | TGG TGG GTG ATG ACG GGA | SEQ ID NO: 43 |

Production of Tranzyme RNA and Post-Transcriptional Cleavage

PCR Production of Linear Expression Templates: To replace the depleted tRNA, genes were designed to be promoted by the T7 promoter and contain a hammerhead ribozyme and sequence encoding for tRNA. The translated region is called a tranzyme, as it includes a ribozyme and the DNA to encode for respective transfer RNA. To facilitate appropriate termination of transcription, linear expression templates (LETs) were produced to contain the T7 promoter and tranzyme gene. The 3′ end of the LET is blunt at the 3′ end of the tranzyme gene, allowing for the RNA polymerase to end transcription in the absence of a RNA polymerase termination sequence. PCR reactions to produce LETs were as follows: 2 μL PFU DNA polymerase, 1 mM dNTPs, 5 mM of each sense and antisense primer, 5-500 pg template plasmid, 20 mM Tris-Cl pH 8.8, 2 mM magnesium sulfate, 10 mM potassium chloride, 10 mM ammonium sulfate, 0.1% v/v Triton™ X-100, and 0.1 μg/mL bovine serum albumin. Following 30 rounds of thermocycling, the PCR reaction is purified and concentrated via ethanol precipitation with a 70% ethanol wash. The resulting DNA is suspended in water for further use.

In vitro Transcription (IVT): tRNA was synthesized via in vitro transcription. Linear expression templates detailed above were combined with T7 RNA polymerase, 5 mM of each ATP, UTP, GTP, and CTP, 5 mM spermadine, 2 mM 1,4-diaminobutane, 800 units/mL murine RNAse Inhibitor (Sigma Aldrich, USA), 15 mM magnesium chloride, 5 mM dithiothreitol, and 50 mM Tris-Cl pH 7.5. After transcription of RNA, the tranzyme sequence is designed to anneal to itself and auto-cleave at the 5′ end of the tRNA sequence, resulting in two cleavage products: 1) hammerhead ribozyme and 2) transfer RNA.

Transcription and Tranzyme Cleavage Analysis: IVT reactions were electrophoresed using TBE-Urea polyacrylamide gel electrophoresis (TU-PAGE). An oligonucleotides ladder was run simultaneously for size comparison. After electrophoresis, gels were washed for 20 minutes in TBE buffer and subsequently stained for 20 minutes in 0.1 μg/mL ethidium bromide. Stained gels were imaged under UV light.

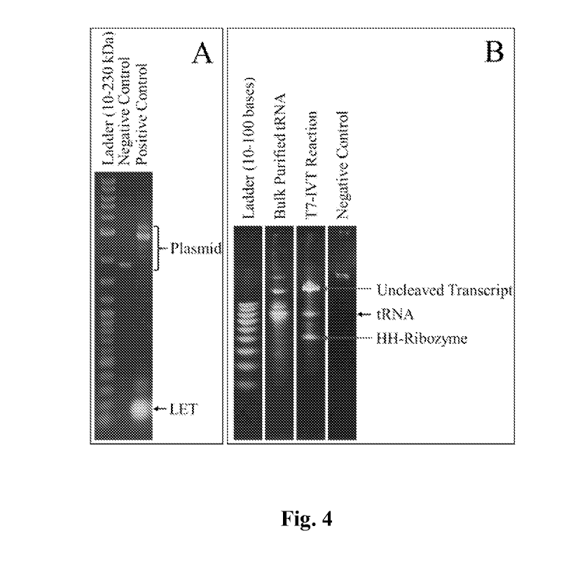

As shown in FIG. 4, PCR reactions yielded LETs of appropriate length. IVT using LETs produced oligonucleotides of expected length. The presence of two distinct cleavage products demonstrates the successful auto-cleavage of the tranzyme RNA.

### Example 3

### Cell-Free Protein Synthesis by Coexpression of Tranzyme RNA in tRNA-Depleted Extract

Example Gene: A test gene was designed to produce poly-valine, with the specific nucleotide sequence

(SEQ ID NO: 44)
ATG-(GTA)₂₀(ATGGTAAGTAGTAGTAGTAGTAGTAGTAGTAGTAGT
AGTAGTAGTAGTAGTAGTAGTAGTAGTA)
for the protein
(SEQ ID NO: 45)
Met-(Val)₂₀(MVVVVVVVVVVVVVVVVVVVV).

CFPS Reaction: Reactions were performed as described in example 1 with RNAse treated extracts and the following modifications. The plasmid added for the gene of interest encoded for the test gene PolyValₙ. LETs encoding for tranzymes of fMetCAU and ValUAC were included in the reaction at 50 and 100 μg/mL, respectively. Radiolabeled 14-C Val was included in the reaction mixture at 5.25 μM. In the negative control case, no tRNA nor tranzyme LETs were added. For the positive control, bulk purified E. coli tRNA was added a 2 μg/mL.

Protein Yield Quantification: Total and incorporated radiolabeled protein was quantified using 5% v/v TCA precipitation and liquid scintillation counting.

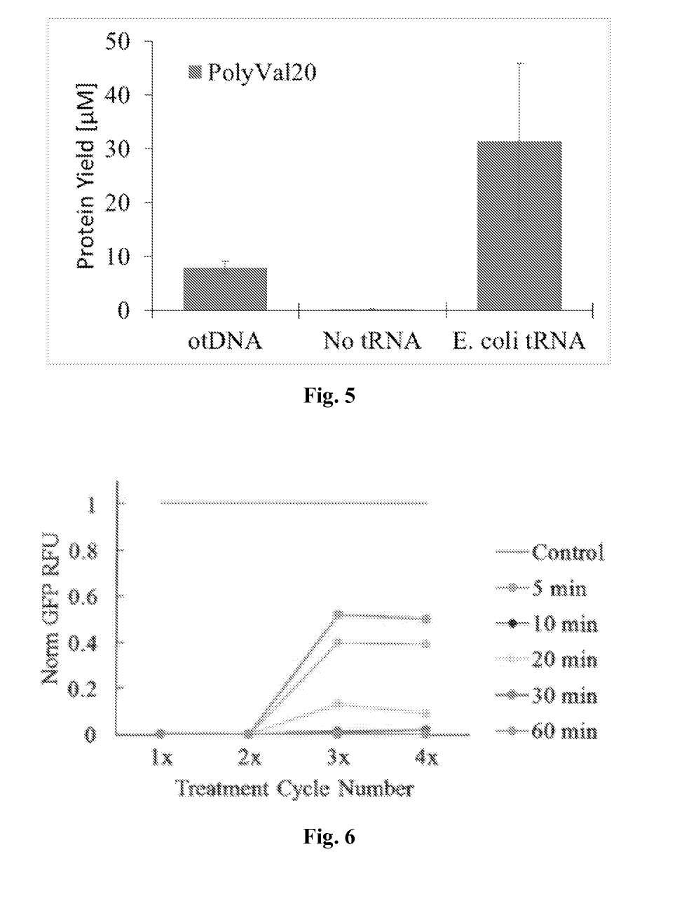

As shown in FIG. 5, the negative control exhibited undetectable levels of protein synthesis. The positive control containing bulk tRNA yielded protein as much as 40 μg/mL. The coexpression of tranzyme RNA (fMet and Val) yielded protein as much as 9 μg/mL.

### Example 4

### Recycle of Magnetic Beads Decorated with RNAse

Recycled Treatment and CFPS Reactions: tRNA depletion of cell-free extract was performed as detailed in example 1. Treatment reactions were repeated 5 times with the same beads. Following each treatment reaction, beads were recovered, suspended, washed and re-aggregated 2 times in 100 μL PBS containing 0.5% v/v Tween20® and 1 time with 100 μL PBS pH 7.4. Beads were then re-suspended in 35 μL PBS and reused or replaced into storage.

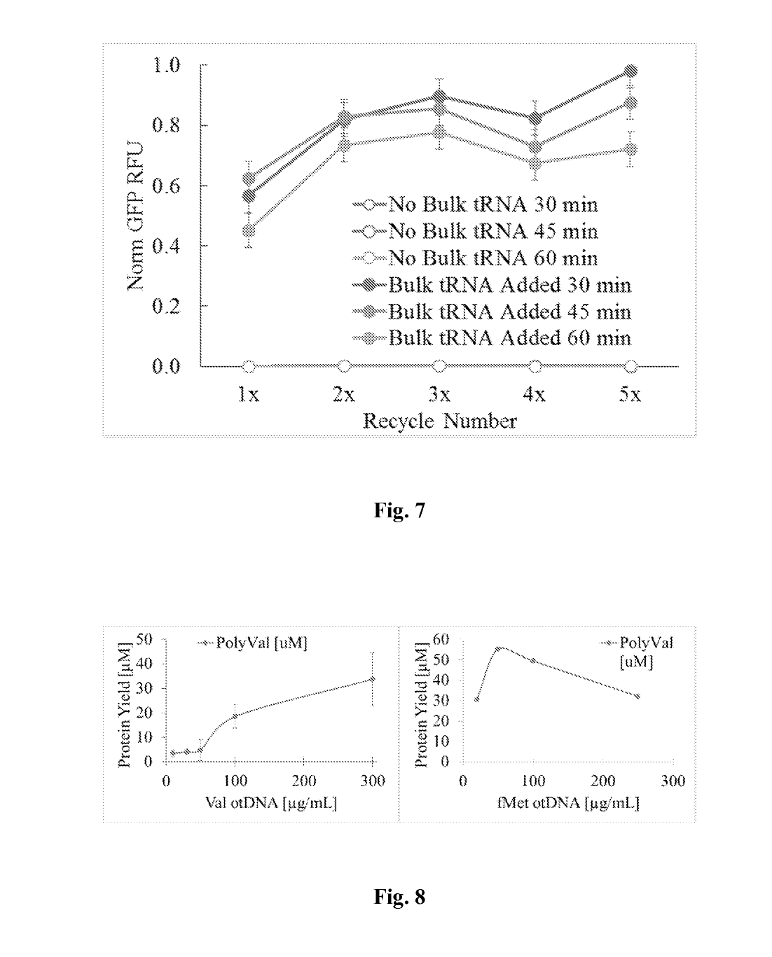

As shown in FIG. 6, the bead-immobilized RNAses can be recycled and no loss in performance of the beads if given sufficient treatment incubation time. As also shown in FIG. 7, after 5 treatment reactions using the same beads, no loss in performance of the beads was observed. Gel electrophoresis of purified nucleic acids from treated extracts suggested degradation of the tRNA. Cell-free reactions of treated extracts showed similar recovery of protein production. This experiment demonstrated the potential to completely replace endogenous tRNA with either exogenous tRNA or synthetic tRNA constructs.

### Example 5

### Optimization of tRNA and Tranzyme RNA for Protein Synthesis

CFPS Results: The PolyVal gene relies on 2 tranzymes for production (fMet and Val). To demonstrate the dependence and optimization of these tranzymes, the levels of one LET (orthogonal tranzyme DNA or otDNA) was varied as the other was held constant. For example, Val otDNA was varied as fMet otDNA was held at 50 μg/mL. The ability to directly control tranzyme template concentrations allows for ready optimization of the protein expression. As shown in FIG. 8, cell-free protein synthesis production yields can be optimized by optimizing the two different tranzyme tRNA gene template concentrations, both of which are needed for the production of Methionine initiated polyvaline.

### Prophetic Examples

### Example 6

### Non-Natural Amino Acid Incorporation in CFPS Using a tRNA-Depleted Extract

Cell-free Protein Synthesis: CFPS of GFP will be performed using tRNA-depleted CPFS reaction conditions as specified in Example 1 with the following modifications: orthogonal amino-acyl tRNA synthetase, orthogonal tranzyme DNA, and a non-natural amino acid will be added to the synthesis reactions. For example, Methanocaldococcus jannaschii TyrRS mutated to accept the non-natural amino acid p-propargyloxyphenylalanine (pPa) and an appropriate Mj tRNA containing the amber stop anticodon will be included in the reaction at optimized concentrations as detailed in Example 5. pPa will be included as well at 2 mM. The Orthogonal tRNA can be produced from appropriate tranzyme genes by in vitro transcription, expression in a cell-free extract or co-expression during cell-free protein synthesis.

The E. coli-orthogonal tRNA synthetase can be overexpressed from the plasmid pEVOL-pPrF harbored in BL21 Star™ (DE3) in shake flasks containing 1 L of 2xYT media at 378 C and 280 rpm. Expression can be induced with 1 mM IPTG at 0.5 OD600 and incubated overnight. Cells can be lysed by Emusiflex B-15 French Press and the synthetase can be purified using HisTrap™ HP columns.

Yield Analysis: In the specific case mentioned above, the amber stop codon will cause the GFP protein to terminate prematurely if the orthogonal tRNA does not present itself. GFP is fluorescent only when full length, therefore GFP fluorescence from CFPS includes proteins that contain pPa.

### Example 7

### Cell-Free Protein Synthesis by Coexpression of 21 Tranzyme RNAs in tRNA-Depleted Extract

Gene Design: A gene for Firefly luciferase is synthesized de novo such that the entirety of the gene is represented by a minimal set of codons, specifically, 20 codons. The start codon (ATG) for the initial methionine requires a specific tRNA (fMet) to initiate peptide elongation in the ribosome. Therefore, 21 tRNA are required to represent 20 codons.

Cell-free Protein Synthesis: CFPS of a Firefly luciferase gene comprised from a minimal set of 20 codons will be performed using tRNA-depleted CFPS reaction conditions as specified in Example 3 with the following modifications: 1) In addition to adding the Val and fMet tranzyme DNA linear expression templates, tranzyme DNA linear expression templates for the remaining 19 amino acids required for the complete proteomic canon will be included at optimized concentrations as detailed in Example 5; 2) the Firefly luciferase gene described above will be added to the reaction to template its production.

Yield Analysis: Bioluminescence will be measured using a monochrometer (i.e. Synergy MX, Biotek, USA) measuring at 570 nm wavelength. In a 96-well plate, 20 μL of CFPS reaction will be mixed with 40 μL ONE-GLO™ Luciferase assay mix (Promega, USA), the plate will shake for 15 seconds and the luminescence will be measured.

### Example 8

### Sense Codon Reassignment for Non-Natural Amino Acid Incorporation

Gene Design: An example gene is designed for the peptide:

(SEQ ID NO: 46)
MYVVVVVVVVV

with the nucleic acid sequence

(SEQ ID NO: 47)
ATG TAC GTA GTA GTA GTA GTA GTA GTA GTA GTA.

The gene could contain more or fewer Val. This gene is similar to the gene expressed in Example 3 but with an insertion of a different codon after the initiating methionine codon. An orthogonal tranzyme, in this case derived from Methanocaldococcus jannaschii, will be designed to contain an appropriate anticodon, in this case the anticodon for the tyrosine codon TAC (GTA).

Cell-free Protein Synthesis: The example gene will be expressed in CFPS using tRNA-depleted CFPS reaction conditions as specified in Example 3 with the following modifications: the extract orthogonal amino-acyl tRNA synthetase, orthogonal tranzyme DNA with the appropriate anticodon, and a non-natural amino acid will be added to the synthesis reactions. For example, Methanocaldococcus jannaschii TyrRS mutated to accept the non-natural amino acid p-propargyloxyphenylalanine (pPa) and an appropriate M. jannaschii tRNA containing the anticodon GUA are included in the reaction. pPa will be included as well at 2 mM. In one specific example peptide, the codon typically reserved for tyrosine (TAC) will be targeted by the orthogonal aminoacyl-tRNA, resulting in a peptide sequence of

(SEQ ID NO: 48)
M-pPa-VVVVVVVVV.

Yield Analysis: C¹⁴-labeled valine and TCA precipitation of the CPFS product followed by scintillation counting to measure radiation will be employed to quantify polyvaline production. Gel electrophoresis and autoradiography can be employed to verify correct product production.

### Example 9

### Probing Codon: Anticodon Promiscuity for Optimal Minimal Codon Set

Gene Design: Codon:anticodon pairing naturally exhibits some amount of promiscuity or degeneracy. To verify that pairs are exclusive and non-promiscuous, test proteins are designed. Test proteins will have the template nucleic acid sequence of

(SEQ ID NO: 49)
ATG-XYZ-(GTA)₂₀(ATGNNNGTAGTAGTAGTAGTAGTAGTAGTAGTA
GTAGTAGTAGTAGTAGTAGTAGTAGTAGTAGTA)

where “NNN” represents the codon being probed for degeneracy. Exemplary pairing of “NNN” is described below using “XYZ” as exemplary codons.

Cell-free Protein Synthesis: CFPS will be performed using tRNA-depleted CFPS reaction conditions as specified in Example 3 with the exception of adding additional tranzyme DNA linear expression templates at optimized concentrations as in Example 5. The test protein will be coexpressed with tranzyme genes for fMetCAU, ValUAC, and the tranzyme of interest. For example, the tranzyme may contain the anticodon “Z′Y′X′”—i.e. a standard Watson-Crick pairing for the codon “XYZ.” To examine degeneracy, the anticodon sequence may be mutated to “Y′Y′X′” for example. Protein expression will be dependent on the existence of sufficient pairing that the “XYZ” codon encodes. The codon:anticodon pairing may be considered degenerate or promiscuous when protein expression using the anticodon “Y′Y′X′” is equal to or nearly equal to expression using “Z′Y′X′.” Codon:anticodon pairing may be considered orthogonal or exclusive when protein expression using the anticodon “Y′Y′X′” is significantly less than expression levels using “Z′Y′X′.”

Yield Analysis: C¹⁴-labeled valine and TCA precipitation of the CPFS product followed by scintillation counting to measure radiation will be employed to quantify polyvaline production. Gel electrophoresis and autoradiography can be employed to verify correct product production.

Although the invention herein has been described in connection with a preferred embodiment thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

### STATEMENT

1. A method for incorporating at least one non-natural amino acid into a polypeptide using a cell-free protein synthesis system, wherein the cell-free protein synthesis system comprises a cell-free extract and is deficient in endogenous tRNA, the method comprising:

providing the synthesis system with the at least one non-natural amino acid, at least one orthogonal aminoacyl-tRNA synthetase and an orthogonal tRNA corresponding to the at least one non-natural amino acid, wherein the orthogonal aminoacyl-tRNA synthetase aminoacylates the corresponding orthogonal tRNA with the non-natural amino acid.

2. The method of claim 1, further comprising:

removing the endogenous tRNA in the cell-free protein synthesis system by treating the synthesis system with a ribonuclease to degrade the endogenous tRNA.

3. The method of claim 1 or 2, further comprising:

providing the synthesis system with a set of tRNAs for one or more natural amino acids and the corresponding amino-acyl tRNA synthetases, wherein each of the one or more natural amino acids corresponds to a single unique codon.

4. The method of claim 3, wherein the set of tRNAs comprises tRNAs for at least 2 natural amino acids.
5. The method of claim 3, wherein the set of tRNAs for one or more natural amino acids are produced in the cell-free protein synthesis system with a tRNA gene as template.
6. The method of any one of claims 1 to 5, further comprising:

selecting codons for the non-natural amino acids based on the codons of the one or more natural amino acids so that the competition between the natural amino acids and the non-natural amino acids to be attached to the orthogonal tRNAs is reduced.

7. The method of claim 6, further comprising:

selecting codons for the non-natural amino acids based on frequency of codon use and frequency of being incorporated by natural tRNAs.

8. The method of any one of claims 1 to 7, wherein the tRNAs for one or more natural or non-natural amino acids are obtained through the steps comprising:

identifying gene sequences encoding for tRNAs for the one or more natural or non-natural amino acids; and

producing the tRNAs for the natural or non-natural amino acids with an RNA polymerase and a tRNA gene template comprising the gene sequences.

9. The method of claim 8, wherein the steps further comprises removing the gene sequences encoding for tRNAs that form wobble base pairing or other non-specific base pairing with the codons of natural amino acids.
10. The method of any one of claims 6 to 7, wherein the RNA polymerase is a T7 RNA polymerase.
11. The method of any one of claims 8 to 10, wherein the tRNA gene template comprises a gene sequence encoding a ribozyme.
12. The method of any one of claims 8 to 11, wherein the tRNA gene template comprises a hammerhead ribozyme.
13. The method of any one of claims 8 to 12, wherein the tRNA gene template comprises a linear tRNA gene template without a transcription terminator sequence.
14. The method of any one of claims 8 to 13, wherein the tRNAs for the natural amino acids are produced by PCR.
15. The method of any one of claims 2 to 14, wherein the ribonuclease is selected from the group consisting of RNase A, Onconase and Colicin D tRNase.
16. The method of any one of claims 2 to 15, wherein the ribonuclease is immobilized on a carrier or surface.
17. The method of any one of claims 2 to 16, wherein the ribonuclease is immobilized onto magnetic beads.
18. The method of any one of claims 2 to 17, wherein the ribonuclease is recycled.
19. The method of any one of claims 1 to 18, wherein the cell-free protein synthesis system is an E. coli-based cell-free protein synthesis system.
20. The method of any one of claims 1 to 19, wherein the orthogonal aminoacyl-tRNA synthetase is derived from one or more of M. jannaschii, M. barkeri, and P. horikoshii.
21. The method of any one of claims 1 to 20, further comprising selecting a concentration of the orthogonal tRNA and the orthogonal aminoacyl-tRNA synthetase to increase efficiency of the non-natural amino acid incorporation.
22. The method of any one of claims 1 to 21, further comprising selecting a concentration of the non-natural amino acid to increase efficiency of the non-natural amino acid incorporation.
23. The method of any one of claims 3 to 22, wherein the set of tRNAs for one or more natural amino acids are produced in the cell-free protein synthesis system with tRNA genes as template, the method further comprising selecting a concentration ratio of the tRNAs and tRNA gene template to increase efficiency of the non-natural amino acid incorporation.
24. A method for producing a set of tRNAs for one or more natural or non-natural amino acids, comprising

identifying gene sequences encoding for tRNAs for the one or more natural or non-natural amino acids;

removing the gene sequences with overlapping anticodon sequences for each of the one or more natural or non-natural amino acid; and

producing the tRNAs for the natural or non-natural amino acids with RNA polymerase and tRNA gene templates comprising the gene sequences.

25. The method of claim 24, further comprising removing the gene sequences encoding for tRNAs that form wobble base pairing or otherwise non-specific base pairing with the codons of natural or non-natural amino acids.
26. The method of any of claims 24 to 25, wherein the tRNA gene templates comprise a linear tRNA gene template without a transcription terminator sequence.
27. The method of any of claims 24 to 26, wherein the tRNA gene templates comprise a gene sequence encoding a ribozyme.
28. The method of any of claims 24 to 27, wherein the tRNA gene templates comprise a hammerhead ribozyme.
29. A set of tRNA gene sequences for one or more natural amino acids comprising a gene sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.
30. A kit for producing a polypeptide with at least one non-natural amino acid incorporated into the polypeptide, comprising a cell-free protein synthesis system wherein endogenous tRNA is removed, at least one non-natural amino acid, at least one orthogonal aminoacyl-tRNA synthetase and its corresponding orthogonal tRNA, wherein the orthogonal aminoacyl-tRNA synthetase preferentially aminoacylates the corresponding orthogonal tRNA with the non-natural amino acid.

What is claimed is:

1. A method for incorporating at least one non-natural amino acid into a polypeptide using a cell-free protein synthesis system, wherein the cell-free protein synthesis system comprises a cell-free extract and is deficient in endogenous tRNA, the method comprising:
providing the synthesis system with the at least one non-natural amino acid, at least one orthogonal aminoacyl-tRNA synthetase and an orthogonal tRNA corresponding to the at least one non-natural amino acid, wherein the orthogonal aminoacyl-tRNA synthetase aminoacylates the corresponding orthogonal tRNA with the non-natural amino acid.

2. The method of claim 1, further comprising:
removing the endogenous tRNA in the cell-free protein synthesis system by treating the synthesis system with a ribonuclease to degrade the endogenous tRNA.

3. The method of claim 1, further comprising:
providing the synthesis system with a set of tRNAs for one or more natural amino acids and the corresponding amino-acyl tRNA synthetases, wherein each of the one or more natural amino acids corresponds to a single unique codon.

4. The method of claim 3, wherein the set of tRNAs comprises tRNAs for at least 2 natural amino acids.

5. The method of claim 3, wherein the set of tRNAs for one or more natural amino acids are produced in the cell-free protein synthesis system with a tRNA gene as template.

6. The method of claim 1, further comprising:
selecting codons for the non-natural amino acids based on the codons of the one or more natural amino acids so that the competition between the natural amino acids and the non-natural amino acids to be attached to the orthogonal tRNAs is reduced.

7. The method of claim 6, further comprising:
selecting codons for the non-natural amino acids based on frequency of codon use and frequency of being incorporated by natural tRNAs.

8. The method of claim 1, wherein the tRNAs for one or more natural or non-natural amino acids are obtained through the steps comprising:
identifying gene sequences encoding for tRNAs for the one or more natural or non-natural amino acids; and
producing the tRNAs for the natural or non-natural amino acids with an RNA polymerase and a tRNA gene template comprising the gene sequences.

9. The method of claim 8, wherein the steps further comprises removing the gene sequences encoding for tRNAs that form wobble base pairing or other non-specific base pairing with the codons of natural amino acids.

10. (canceled)

11. The method of claim 8, wherein the tRNA gene template comprises a gene sequence encoding a ribozyme.

12. (canceled)

13. The method of claim 8, wherein the tRNA gene template comprises a linear tRNA gene template without a transcription terminator sequence.

14. (canceled)

15. The method of claim 2, wherein the ribonuclease is selected from the group consisting of RNase A, Onconase and Colicin D tRNase.

16. (canceled)

17. The method of claim 2, wherein the ribonuclease is immobilized onto magnetic beads.

18. The method of claim 2, wherein the ribonuclease is recycled.

19. The method of claim 1, wherein the cell-free protein synthesis system is an E. coli-based cell-free protein synthesis system.

20. The method of claim 1, wherein the orthogonal aminoacyl-tRNA synthetase is derived from one or more of M. jannaschii, M. barkeri, and P. horikoshii.

21. The method of claim 1, further comprising selecting a concentration of the orthogonal tRNA and the orthogonal aminoacyl-tRNA synthetase to increase efficiency of the non-natural amino acid incorporation and further comprising selecting a concentration of the non-natural amino acid to increase efficiency of the non-natural amino acid incorporation.

22. (canceled)

23. The method of claim 3, wherein the set of tRNAs for one or more natural amino acids are produced in the cell-free protein synthesis system with tRNA genes as template, the method further comprising selecting a concentration ratio of the tRNAs and tRNA gene template to increase efficiency of the non-natural amino acid incorporation.

24-28. (canceled)

29. A set of tRNA gene sequences for one or more natural amino acids comprising a gene sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

30. A kit for producing a polypeptide with at least one non-natural amino acid incorporated into the polypeptide, comprising a cell-free protein synthesis system wherein endogenous tRNA is removed, at least one non-natural amino acid, at least one orthogonal aminoacyl-tRNA synthetase and its corresponding orthogonal tRNA, wherein the orthogonal aminoacyl-tRNA synthetase preferentially aminoacylates the corresponding orthogonal tRNA with the non-natural amino acid.
